(19) United States
(12) Patent Application Publication
(10) Pub. No.: US 2026/0144598 A1
(43) Pub. Date: May 28, 2026

(54) CALIBRATION METHOD FOR TRACKING SYSTEM IN COMPUTER-ASSISTED SURGERY

(71) Applicant: ORTHOSOFT ULC, Montreal (CA)

(72) Inventors: Louis-Philippe AMIOT, Montreal (CA); Rodolphe RUBRECHT, Mauguio (FR); Yann FACCHINELLO, Prévost (CA); Benoist LEMAITRE, Mauguio (FR); Marc-Antoine DUFOUR, Montreal (CA); Jean-Michel GARIEPY, Mauguio (FR)

(21) Appl. No.: 19/402,400

(22) Filed: Nov. 26, 2025

Related U.S. Application Data
(60) Provisional application No. 63/725,956, filed on Nov. 27, 2024.

(51) Int. Cl. A61B 34/20; A61B 34/00; A61B 34/10; A61B 34/37

(52) U.S. Cl. CPC A61B 34/20; A61B 34/10; A61B 34/25; A61B 34/37; A61B 2034/107; A61B 2034/2055; A61B 2034/2061; A61B 2034/252

(57) ABSTRACT

A system for tracking an instrument in computer-assisted surgery, comprising: a processing unit; and a non-transitory computer-readable memory communicatively coupled to the processing unit and comprising computer-readable program instructions executable by the processing unit for: obtaining intraoperatively at least one image of an instrument having a tracker thereon, the tracker having optical elements thereon arranged in a given pattern; calibrating the instrument by image processing the at least one image to record the given pattern relative to the instrument; and tracking the instrument optically after the calibrating by obtaining images of the given pattern of optical elements.

### CROSS-REFERENCE TO RELATED APPLICATION

The present application claims the benefit of U.S. Patent Application No. 63/725,956, filed on Nov. 27, 2024, the contents of which are incorporated herein by reference.

### TECHNICAL FIELD

The present application relates to computer-assisted surgery including bone and tool tracking, and to the calibration of instruments in the context of computer-assisted surgery, including robotized computer-assisted surgery.

### BACKGROUND OF THE ART

Tracking of surgical instruments or tools is an integral part of computer-assisted surgery (hereinafter “CAS”), including robotized CAS. The end effector, the tools, bodily parts are tracked for position and/or orientation using computerized components in such a way that relative navigation information pertaining to bodily parts is obtained. The information is then used in various interventions (e.g., orthopedic surgery, neurological surgery) with respect to the body, such as bone alterations, implant positioning, incisions and the like during surgery.

In CAS, optical tracking is commonly used in different forms, for instance by the presence of optically-detectable trackers on the end effector and/or operating end of a robotic arm, in addition to being optionally present on the patient. For example, the optically-detectable trackers are active or passive retroreflective components on the robot, on tools and bones, though other types of trackers may be used. The trackers are viewed by a tracking device, such as a tracking system or tracker (e.g., Navitracker®), a depth camera, and by triangulation the position and orientation of the tracker device is calculable to output navigation data. In robotized CAS, the robot arm may also be equipped with a tracker device.

In order to contribute to the precision and accuracy, tools (a.k.a., instruments, surgical instruments, etc) having trackers thereon may be calibrated, with some additional steps optionally done intra-operatively, or peri-operatively. To streamline the surgical procedure, in some instances a calibration file may exist for a tracker, in a concept known as a permanent calibration. The calibration file is retrievable by the CAS system for use during a surgical procedure. The calibration file may consist in a geometric relation between various optical elements of a tracker. As the CAS system will recognize a tracker by way of the geometric relation between the optical elements, the access to the calibration file may be central to the tracking operation.

Additional calibration steps may then be required. For example, the geometric relation between the tracking and the working end of the tool may need to be recorded, such that the subsequent tracking of the tracker enables the CAS system to output navigation data for the tool, which navigation data is associated with the working end of the tool. The working end of the tool may be a tip and/or axis of a registration pointer, the blade of a saw, the reaming end of a reamer and a rotational axis thereof, as examples among others.

The calibration file may be based on manufacturer models of the trackers. Typically, the tracker includes a support for optical elements, and the manufacturer models include a geometry of the support. Because of various factors, the physical version of the support may not be an exact match with the geometry of the support in the manufacturer model, such as because of deformation resulting from the demolding, temperature variations, tolerances, etc.

### SUMMARY

In accordance with a first aspect of the present disclosure, there is provided a system for tracking an instrument in computer-assisted surgery, comprising: a processing unit; and a non-transitory computer-readable memory communicatively coupled to the processing unit and comprising computer-readable program instructions executable by the processing unit for: obtaining intraoperatively at least one image of an instrument having a tracker thereon, the tracker having optical elements thereon arranged in a given pattern; calibrating the instrument by image processing the at least one image to record the given pattern relative to the instrument; and tracking the instrument with the tracker optically after the calibrating, by obtaining images of the given pattern of optical elements.

Further in accordance with the first aspect, for example, the tracker has three optical elements thereon, and the given pattern is a scalene triangle.

Still further in accordance with the first aspect, for example, the tracker is a multifaceted tracker, the multifaceted tracker having at least two sets of three optical elements, each of the two sets forming a geometrical pattern, wherein the at least one image includes at least two of the at least two sets of three optical elements.

Still further in accordance with the first aspect, for example, calibrating the instrument includes image processing the at least one image including the at least two of the at least two sets of three optical elements to record a geometric relation between the at least two sets of three optical elements.

Still further in accordance with the first aspect, for example, calibrating the instrument includes image processing the at least one image including the at least one of the at least two sets of three optical elements to record a dimension of at least one of the sets of three optical elements.

Still further in accordance with the first aspect, for example, the multifaceted tracker has three sets of three optical elements.

Still further in accordance with the first aspect, for example, obtaining at least one image of an instrument includes obtaining an image including the three sets of three optical elements.

Still further in accordance with the first aspect, for example, obtaining at least one image of an instrument includes obtaining at least a first image including a first and a second of the sets of three optical elements; obtaining at least a second image including a second and a third of the sets of three optical elements; and obtaining at least a third image including a first and a third of the sets of three optical elements.

Still further in accordance with the first aspect, for example, calibrating the instrument further includes recording a geometrical relation between the tracker and a working end of the instrument.

Still further in accordance with the first aspect, for example, recording the geometrical relation between the tracker and the working end of the instrument is performed by obtaining a model of the instrument and merging the model with the at least one image.

Still further in accordance with the first aspect, for example, recording the geometrical relation between the tracker and the working end of the instrument is performed by obtaining images of the instrument during a given sequence of movement.

Still further in accordance with the first aspect, for example, a calibration file may be retrieved for the instrument, and wherein recording the given pattern includes adjusting values of the calibration file.

Still further in accordance with the first aspect, for example, a display on a graphical user interface may be output showing a movement required to orient and/or position the instrument with the tracker to obtain said at least one image.

Still further in accordance with the first aspect, for example, a display may be output on a graphical user interface showing a position of the instrument with the tracker relative to a calibration volume in which the calibrating occurs.

Still further in accordance with the first aspect, for example, tracking the instrument optically after the calibrating includes tracking the instrument using a first of the at least two sets of optical elements, and switching to tracking the instrument using a second of the at least two sets of optical elements when a line of sight between the first of the at least two sets of optical element and a tracking device is disrupted.

Still further in accordance with the first aspect, for example, obtaining at least one image includes obtaining a video feed.

Still further in accordance with the first aspect, for example, the optical elements are retroreflective members, and wherein the three optical elements of a first of the sets are in a first orientation, the three optical elements of a second of the sets are in a second orientation.

Still further in accordance with the first aspect, for example, the obtaining, calibrating and tracking are performed intraoperatively.

Still further in accordance with the first aspect, for example, a calibration file for the tracker is retrieved prior to the obtaining.

Still further in accordance with the first aspect, for example, the calibration file is updated with the calibrating, for subsequent use.

Still further in accordance with the first aspect, for example, the system may automatically detect a degradation in tracking accuracy, and prompting a recalibration.

In accordance with a second aspect of the present disclosure, there is provided a method for tracking an instrument in computer-assisted surgery, comprising: obtaining intraoperatively at least one image of an instrument having a tracker thereon, the tracker having optical elements thereon arranged in a given pattern; calibrating the instrument by image processing the at least one image to record the given pattern relative to the instrument; and tracking the instrument with the tracker optically after the calibrating, by obtaining images of the given pattern of optical elements.

Further in accordance with the second aspect, for example, the tracker is a multifaceted tracker, the multifaceted tracker having at least two sets of three optical elements, each of the two sets forming a geometrical pattern, wherein the at least one image includes at least two of the at least two sets of three optical elements, and wherein calibrating the instrument includes image processing the at least one image including the at least two of the at least two sets of three optical elements to record a geometric relation between the at least two sets of three optical elements.

Still further in accordance with the second aspect, for example, calibrating the instrument includes image processing the at least one image including the at least one of the at least two sets of three optical elements to record a dimension of at least one of the sets of three optical elements.

Still further in accordance with the second aspect, for example, the multifaceted tracker has three sets of three optical elements, and wherein obtaining at least one image of an instrument includes obtaining an image including the three sets of three optical elements.

Still further in accordance with the second aspect, for example, the multifaceted tracker has three sets of three optical elements, and wherein obtaining at least one image of an instrument includes obtaining at least a first image including a first and a second of the sets of three optical elements; obtaining at least a second image including a second and a third of the sets of three optical elements; and obtaining at least a third image including a first and a third of the sets of three optical elements.

Still further in accordance with the second aspect, for example, calibrating the instrument further includes recording a geometrical relation between the tracker and a working end of the instrument.

Still further in accordance with the second aspect, for example, recording the geometrical relation between the tracker and the working end of the instrument is performed by obtaining a model of the instrument and merging the model with the at least one image.

Still further in accordance with the second aspect, for example, recording the geometrical relation between the tracker and the working end of the instrument is performed by obtaining images of the instrument during a given sequence of movement.

Still further in accordance with the second aspect, for example, a calibration file may be retrieved for the instrument, and wherein recording the given pattern includes adjusting values of the calibration file.

Still further in accordance with the second aspect, for example, a display may be output on a graphical user interface showing a movement required to orient and/or position the instrument with the tracker to obtain said at least one image.

Still further in accordance with the second aspect, for example, a display may be output on a graphical user interface showing a position of the instrument with the tracker relative to a calibration volume in which the calibrating occurs.

Still further in accordance with the second aspect, for example, the tracker is a multifaceted tracker, the multifaceted tracker having at least two sets of three optical elements, each of the two sets forming a geometrical pattern, wherein the at least one image includes at least two of the at least two sets of three optical elements, and wherein tracking the instrument optically after the calibrating includes tracking the instrument using a first of the at least two sets of optical elements, and switching to tracking the instrument using a second of the at least two sets of optical elements when a line of sight between the first of the at least two sets of optical element and a tracking device is disrupted.

Still further in accordance with the second aspect, for example, obtaining at least one image includes obtaining a video feed.

Still further in accordance with the second aspect, for example, the obtaining, calibrating and tracking are performed intraoperatively.

Still further in accordance with the second aspect, for example, a degradation in tracking accuracy may be automatically detected, prompting a recalibration.

### DESCRIPTION OF THE DRAWINGS

FIG. 1 is a schematic view of a CAS system in accordance with an aspect of the present disclosure, relative to a patient;

FIG. 2 is a block diagram of the tracking system for robotized computer-assisted surgery of FIG. 1;

FIG. 3A is a perspective view of an exemplary tracker that may be calibrated for use with the CAS system of FIG. 1;

FIG. 3B is a perspective view of another exemplary tracker that may be calibrated for use with the CAS system of FIG. 1;

FIG. 4A is a perspective view of the tracker of FIG. 3A with tracking patterns exposing errors relative to a calibration file;

FIG. 4B is a perspective view of the tracker of FIG. 4A with calibrated tracking pattern;

FIG. 5 is a graphical user interface that may be used to calibrate the tracking of FIG. 3; and

FIG. 6 is a flow chart of a method for calibrating a tracker in accordance with an aspect of the present disclosure.

### DETAILED DESCRIPTION

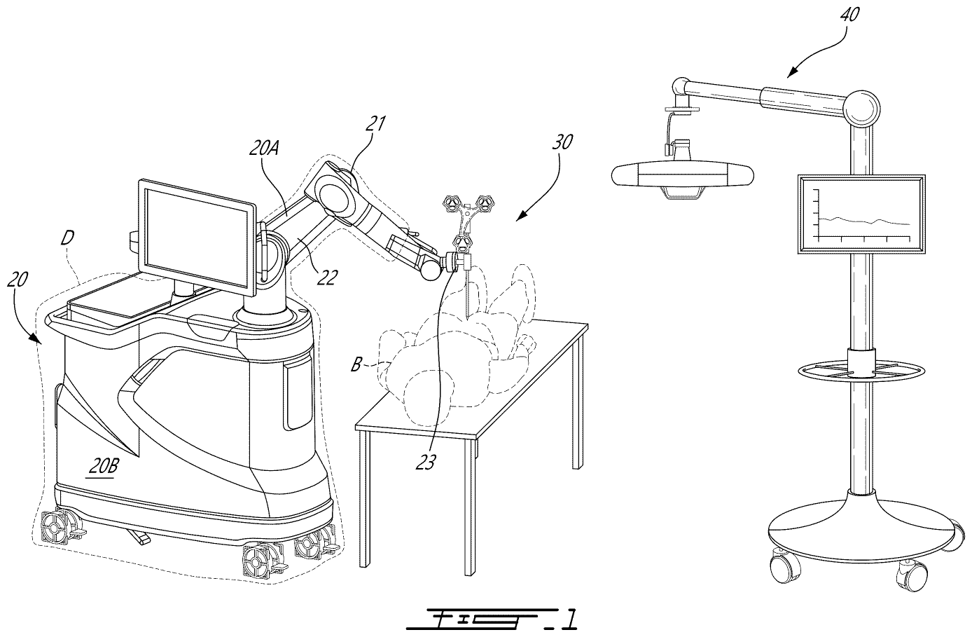

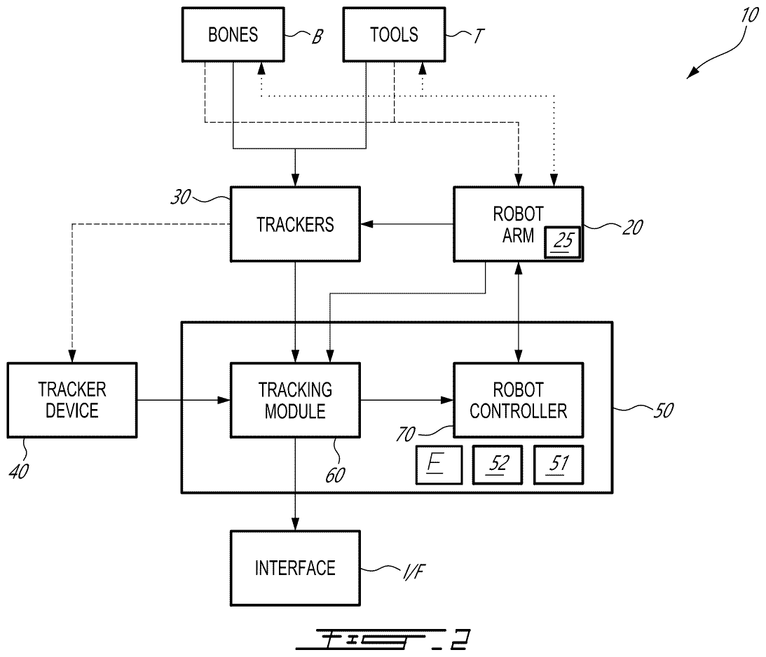

Referring to FIGS. 1 and 2, a computer-assisted surgery (CAS) system is generally shown at 10, and is used to provide surgery assistance to an operator. For simplicity, it will be referred to herein as the system 10. In FIG. 1, the system 10 is shown as being a robotized CAS system 10, but the presence of a robot is optional. Moreover, in FIG. 1, the system 10 is shown relative to a dummy patient in prone decubitus, but only as an example. The system 10 could be used for any body parts, including non-exhaustively hip joint, spine, and shoulder bones, for orthopedic surgery, but could also be used in other types of surgery. For example, the system 10 could be used for surgery of all sorts, such as brain surgery, and soft tissue surgery.

The CAS system 10 may be robotized in a variant, and has, may have or may be used with a robot 20, optical trackers 30, a tracker device 40, a CAS controller 50 (also known as a super controller 50), a tracking module 60, and a robot controller 70 (also known as a robot driver), or any combination thereof:
The robot 20, shown by its robot arm 20A may optionally be present as the working end of the system 10, and may be used to perform or guide bone alterations as planned by an operator and/or the CAS controller 50 and as controlled by the CAS controller 50. The robot arm 20A may also be configured for collaborative/cooperative mode in which the operator may manipulate the robot arm 20, or the tool supported by the robot arm 20, though the tool may be operated by a human operator. For example, the tooling end, also known as end effector, may be manipulated by the operator while supported by the robot arm 20A. The robot 20 may be the coordinate measuring machine (CMM) of the CAS system 10;
The optical trackers 30 are positioned on the robot 20, on patient tissue (e.g., bones B), and/or on the tool(s) T and surgical instruments, and provide tracking data for the robot 20, the patient and/or tools.
The tracking device 40, also known as a sensor device, apparatus, etc performs optical tracking of the optical trackers 30, so as to enable the tracking in space (a.k.a., navigation) of the robot 20, the patient and/or tools;
The CAS controller 50, also known as the super controller, includes the processor(s) and appropriate hardware and software to run a computer-assisted surgery procedure in accordance with one or more workflows. The CAS controller 50 may include or operate the tracking device 40, the tracking module 60, and/or the robot controller 70. As described hereinafter, the CAS controller 50 may also drive the robot arm 20A through a planned surgical procedure;
The tracking module 60 is tasked with determining the position and/or orientation of the various relevant objects during the surgery procedure, such as the end effector of the robot arm 20, bone(s) B and tool(s) T, using data acquired by the tracking device 40 and by the robot 20, and/or obtained from the robot controller 70. The position and/or orientation may be used by the CAS controller 50 to control the robot arm 20A;
The robot controller 70 is tasked with powering or controlling the various joints of the robot arm 20A, based on operator demands or on surgery planning, and may also be referred to as a robot controller module that is part of the super controller 50. The robot controller 70 may also optionally calculate robot movements of the robot arm 20A, so as to control movements of the robot arm 20A autonomously in some instances, i.e., without intervention from the CAS controller 50;
An additional camera(s) may be present, for instance as a complementary registration tool. The camera may for instance be mounted on the robot 20A, such as on the robot arm, such that the point of view of the camera is known in the frame of reference, also known as the coordinate system.

Other components, devices, systems, may be present, such as surgical instruments and tools T, interfaces I/F such as displays, screens, computer station, servers, and like etc. Secondary tracking systems may also be used for redundancy.

Referring to FIG. 1, the robot 20 may have the robot arm 20A stand from a base 20B, for instance in a fixed relation relative to the operating-room (OR) table supporting the patient, whether it is attached or detached from the table. The robot arm 20A has a plurality of joints 21 and links 22, of any appropriate form, to support an end effector 23 that may interface with the patient, or may be used during surgery without interfacing with the patient. For example, the end effector or tool head may optionally incorporate a force/torque sensor for collaborative/cooperative control mode, in which an operator manipulates the robot arm 20A. The robot arm 20A is shown being a serial mechanism, arranged for the tool head 23 to be displaceable in a desired number of degrees of freedom (DOF). The tool head 23 may for example be a support that is not actuated, the support being used to support a tool, with the robot arm 20A used to position the tool relative to the patient. In a variant, the robot arm 20A controls 6-DOF movements of the tool head, i.e., X, Y, Z in the coordinate system, and pitch, roll and yaw. Fewer or additional DOFs may be present. For simplicity, only a fragmented illustration of the joints 21 and links 22 is provided, but more joints 21 of different types may be present to move the end effector 23 in the manner described above. The joints 21 are powered for the robot arm 20A to move as controlled by the CAS controller 50 in the six DOFs, and in such a way that the position and orientation of the end effector 23 in the coordinate system may be known, for instance by readings from encoders on the various joints 21. Therefore, the powering of the joints is such that the end effector 23 of the robot arm 20A may execute precise movements, such as moving along a single direction in one translation DOF, or being restricted to moving along a plane, among possibilities. Such robot arms 20A are known, for instance as described in U.S. patent application Ser. No. 11/610,728, and incorporated herein by reference.

The end effector 23 of robot arm 20A may be defined by a chuck or like tool interface, typically actuatable in rotation. As a non-exhaustive example, numerous tools may be used as end effector for the robot arm 20, such tools including a registration pointer as shown in FIG. 1, equipped with a tracker device 30, a reamer (e.g., cylindrical, tapered), a reciprocating saw, a retractor, a camera, an ultrasound unit, a laser rangefinder or light-emitting device (e.g., the indicator device of U.S. Pat. No. 8,882,777), a laminar spreader, an instrument holder, or a cutting guide, depending on the nature of the surgery. The various tools may be part of a multi-mandible configuration or may be interchangeable, whether with human assistance, or as an automated process. The installation of a tool in the tool head may then require some calibration in order to track the installed tool in the X, Y, Z coordinate system of the robot arm 20.

The end effector 23 of the robot arm 20A may be positioned by the robot 20 relative to surgical area A in a desired orientation according to a surgical plan, such as a plan based on preoperative imaging. Due to the proximity between the robot 20 and the surgical area A, the robot 20 may be covered partially with a surgical drape D, also known as a surgical robotic drape. The surgical drape D is a sterile panel (or panels), tubes, bags or the like that form(s) a physical barrier between the sterile zone (e.g., surgical area) and some equipment that may not fully comply with sterilization standards, such as the robot 20. In an embodiment, the surgical drape D is transparent such that one can see through the drape D. In an embodiment, the robot is entirely covered with the surgical drape D, and this includes the base 20B, but with the exception of the end effector 23. Indeed, as the end effector 23 interacts or may interact with the human body, it may be sterilized and may not need to be covered by the surgical drape D, to access the patient. Some part of the robot 20 may also be on the sterile side of the surgical drape D. In a variant, a portion of the robot arm 20 is covered by the surgical drape D. For example, the surgical drape D may be in accordance with U.S. patent application Ser. No. 15/803,247, filed on Nov. 3, 2017 and incorporated herein by reference.

In order to position the end effector 23 of the robot arm 20A relative to the patient B, the CAS controller 50 can manipulate the robot arm 20A automatically (without human intervention), or by a surgeon manually operating the robot arm 20A (e.g. physically manipulating, via a remote controller through the interface I/F) to move the end effector 23 of the robot arm 20A to the desired location, e.g., a location called for by a surgical plan to align an instrument relative to the anatomy. Once aligned, a step of a surgical procedure can be performed, such as by using the end effector 23. To assist in the maneuvering and navigating of the robot arm 20A, a tracker device 30 may optionally be secured to the distalmost link, and may be distinct from the tracker device 30 on the instrument supported by the end effector 23.

As shown in FIG. 2, the robot arm 20A may include sensors 25 in its various joints 21 and links 22. The sensors 25 may be of any appropriate type, such as rotary encoders, optical sensors, position switches that are a non-exhaustive list of potential sensors, for the position and orientation of the end effector, and of the tool in the end effector 23 to be known. More particularly, the tracking module 60 may determine the position and orientation of the robot 20 in a frame of reference of the robot 20, such as by obtaining the position (x, y, z) and orientation (phi, theta, ro) of the end effector 23 from the CAS controller 50 using the sensors 25 in the robot arm 20A, i.e., robot coordinates may be an integrated function of the robot 20 in that it may determine the position and orientation of its end effector 23 with respect to its coordinate system. Using the data from the sensors 25, the robot 20 may be the coordinate measuring machine (CMM) of the CAS system 10, with a frame of reference (e.g., coordinate system, referential system) of the procedure being relative to the fixed position of the base 20B of the robot 20. The sensors 25 must provide the precision and accuracy appropriate for surgical procedures. The coupling of tools to the robot arm 20A may automatically cause a registration of the position and orientation of the tools in the frame of reference of the robot 20, though steps of calibration could be performed, as explained below.

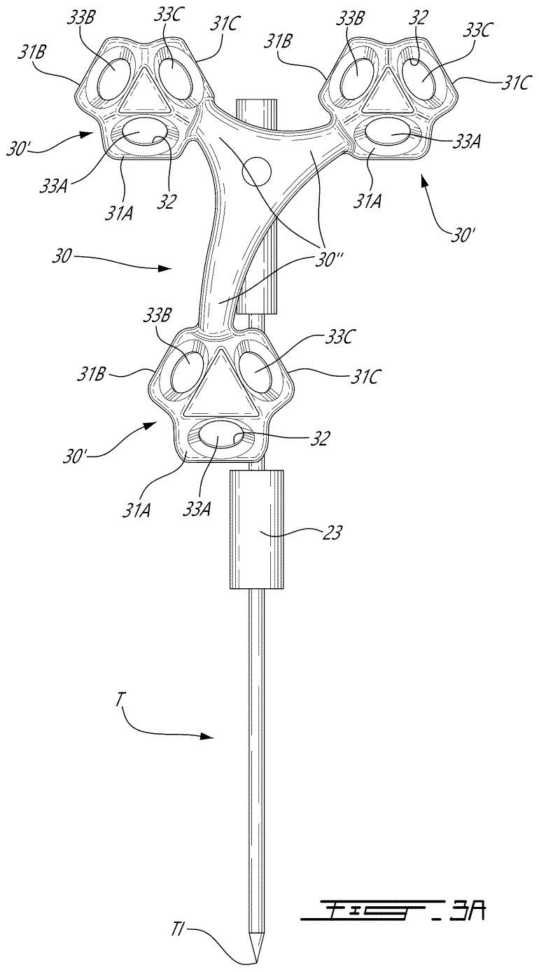

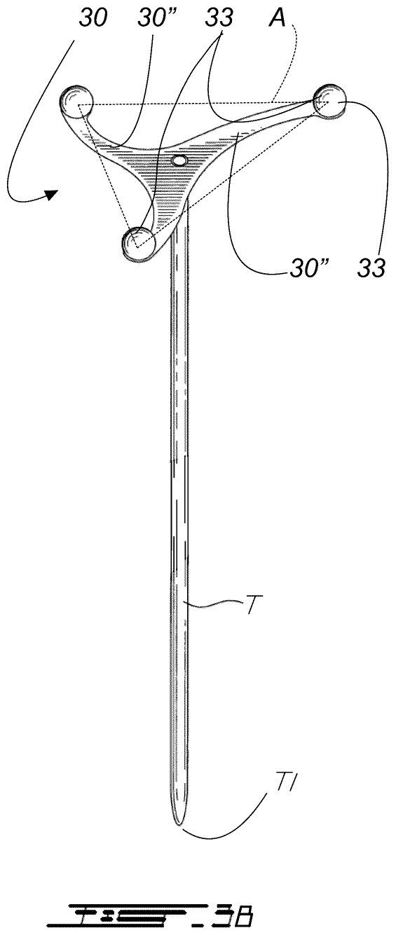

Referring to FIG. 1, an exemplary tracker 30 is shown secured to the instrument T and at the end effector 23 on the robot 20, and may also or alternatively be on the robot arm 20 and/or on the bones B. The trackers 30 may be known as trackable elements, markers, navigation markers, active sensors (e.g., wired or wireless) that may for example include infrared emitters, light-emitting diodes (LEDs). The trackers 30 may also be referred to as a tracker device, to be tracked by the tracking device 40, i.e., the camera. In a variant, the trackers 30 are passive retro-reflective elements, that reflect light. The trackers 30 have optical elements that are arranged in a specific geometrical pattern so as to be recognizably through optical detection by the tracker device 40. For example, the trackers 30 may be retro-reflective lenses. Referring to FIG. 3A, an exemplary tracker 30 is shown as being mounted to a registration pointer T having a tip T1. This is one of numerous tools that may be equipped with the tracker 30. The tracker 30 of FIG. 3A, may be as described in U.S. Pat. No. 8,386,022. Another exemplary tracker 30 is shown in FIG. 3B, and is another example of a tracker 30 that may be used with the CAS system 10 and calibrated in accordance with the present disclosure.

The tracker 30 of FIG. 3A may thus be known as a multifaceted tracker. The tracker 30 of the exemplary embodiment of FIG. 3A has a support for optical elements. The support may have three tracker ends 30′ supported by arms 30″ that interface the tracker ends 30′ to the instrument T. Each tracker end 30′ is provided in three sets of three detectable elements. For example, the tracker ends 30′ are each provided with a pyramidal body having faces 31A, 31B and 31C (hereinafter faces 31 unless otherwise indicated). The faces 31 each define an opening 32 having a given geometrical shape. In the embodiment of FIG. 3, the given geometrical shape is a circle.

Retro-reflective surfaces are positioned in the openings 32, so as to form circular optical elements 33A, 33B, and 33C, respectively provided in the faces 31A, 31B, and 31C of the tracker ends 30'. Other shapes are also considered for the optical elements 33. The retro-reflective surfaces are made of a retro-reflective material that will be detected by the optical tracker device 40 associated with the CAS system 10. For instance, the material Scotch Lite™ is suited to be used as retro-reflective surface.

As the optical elements 33 must be in a given geometrical pattern to be recognized by the optical tracker device 40 of the CAS system 10, the optical elements 33 are regrouped in one embodiment in sets of three. Referring to FIG. 1, a first set of three elements 33 consists of the optical elements 33A, each of which is in a different one of the tracker ends 30′, thereby forming geometrical pattern A. Similarly, a second set consists of the elements 33B forming geometrical pattern B, and a third set consists of the elements 33C forming geometrical pattern C. There may be a single set of elements 33, a pair of sets of elements 33, or more than the three sets of elements 33 illustrated.

In the embodiment of FIG. 3A, each of the elements of a same set (e.g., the first set of elements 33A) are parallel to a same plane, though this exact geometrical relation may not be necessary. Accordingly, the elements 33A are visible from a same field of view. The sets of elements 33 are strategically positioned with respect to one another so as to optimize a range of visibility of the tracker device 10. More specifically, the sets are positioned such that once the tracker device 40 of the CAS system 10 loses sight of one of the sets, another set may be visible. This ensures the continuous tracking of the tool T having a tracker device 30 within a given range of field of view.

The sets each form a geometrical pattern that is recognized by the tracking module 60 of the CAS system 10, i.e., geometrical patterns A, B and C. The combination of circular openings 32 and retro-reflective surface gives a circular shape to the optical elements 33. According to the angle of view of the tracker device 40, these circles will not always appear as being circular in shape. Therefore, the position of the center of the circles can be calculated as a function of the shape perceived from the angle of view by the optical sensor apparatus.

In the embodiment of FIG. 3A, the geometrical patterns A, B, C therefore each consist of a triangle defined by the centers of the optical elements 33 of the sets, with the triangles A, B and C each being a scalene triangle. It is suggested that the three triangles of the three different sets of optical elements 33 be of different shape, with each triangle being associated with a specific orientation with respect to the tool. Alternatively, the three triangles formed by the three different sets may be the same, but the perceived shape of the circular reflective surfaces 33 must be used to identify which of the three sets of reflective surfaces 33 is seen. There may be more or less optical elements, and sets of optical elements, as described in U.S. Pat. No. 8,386,022. Moreover, although triangular geometrical patterns are illustrated, it is contemplated to use other geometrical patterns, such as lines and various polygonal shapes.

The tracker 30 of FIG. 3A may have a calibration file F (FIG. 2). The calibration file F is a digital record of the specific details of the geometrical patterns of the tracker 30, e.g., the length and angles of the triangles A, B and C. The calibration file F may be part of a read-only memory of the CAS system 10, or may be retrievable. For this purpose, the tracker device 30 may have an identifier, for the calibration file F specific to the model of the tracker 30 to be retrieved. In a variant, the calibration file F may not be modified. The calibration file F may be based on a manufacturer 3D model of the tracker 30.

The calibration file F may include other data, such as a geometrical relation between the tracker 30 and the instrument T that supports it, in instances in which the instruments T come with a dedicated tracker 30. Therefore, an example of geometrical relation that may be part of the calibration file F includes a position of a working tip, a position and orientation of an axis, etc, of the instrument T relative to the geometrical patterns A, B, C (if A, B and C are present). As an alternative, the geometrical relation between the instrument T and the tracker 30 may be defined during a calibration step intraoperatively or peri-operatively. The calibration file F may be regarded as a permanent calibration, i.e., one that can be used repeatedly for the tracker 30.

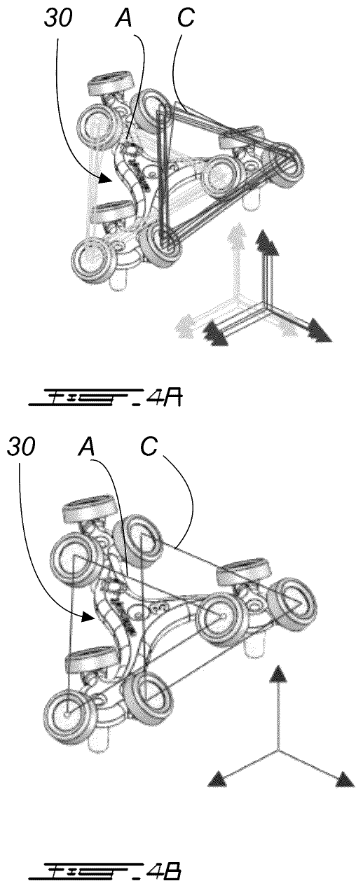

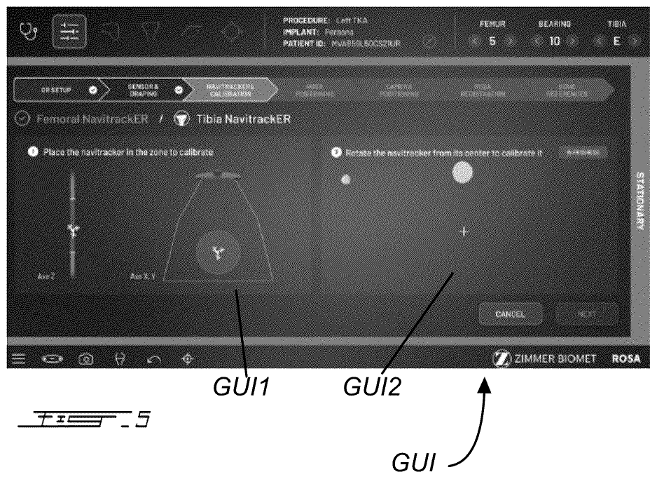

Referring to FIG. 4A, an example of the tracker 30 is shown, but with a multitude of geometrical patterns A and C (the geometrical patterns B are removed for clarity), illustrative of a variability in the location of the geometrical patterns A, B and C in physical versions of the tracker 30 and/or of a variability in the geometry of patterns A, B and/or C. FIG. 4A is therefore also representative of a discrepancy between the calibration file F and the physical version of the tracker 30. It can be observed that, for a same model of the tracker 30, the actual location and/or size of the geometrical patterns A, B and/or C may vary. This may be due to various factors, such as tolerances in manufacturing, deformation resulting from fabrication process, temperature variations, deformation caused by sterilization, etc. As the geometrical patterns in the calibration file F are part of the tracking calculations, the discrepancy between the calibration file F and the physical version of the tracker 30 may result in a loss of accuracy and/or precision in the calculation of tracking data. This is shown by the referential systems also in FIG. 4A, indicating that the discrepancy may have an impact on the location of the tracker 30 in the referential system of the CAS system 10.

Thus, a calibration of the surgical tool with the tracker 30 of FIG. 3A or FIG. 3B, is preferably performed prior to the use of the tool T, to calibrate a position and/or orientation of each of the detectable geometrical patterns to one another, to set (i.e., record) the dimensional data of the geometrical patterns A, B, and C, relative to one another. The calibration of the tool T may also be used to set the geometrical data of the tracker 30 with respect to the tool T, in the manner explained below, and thus locate the geometrical patterns A, B and C, in the manner shown in FIG. 4B. The calibration may be done perioperatively or intra-operatively, or in a manufacturing setting. This will be explained hereinbelow.

Referring to FIG. 3B, the tracker 30 of another exemplary embodiment also has a support for optical elements. The support may have three arms 30″ that interface the optical elements 33 to the instrument T. The optical elements 33 are balls, i.e., truncated spheres. The optical elements may be made of or covered by a retro-reflective material that will be detected by the optical tracker device 40 associated with the CAS system 10. For instance, the material Scotch Lite™ is suited to be used as retro-reflective surface.

As the optical elements 33 must be in a given geometrical pattern to be recognized by the optical tracker device 40 of the CAS system 10, the optical elements 33 are regrouped in one embodiment in a set of three, through there may be more or fewer than the three optical elements 33.

The optical element 33 form a geometrical pattern that is recognized by the tracking module 60 of the CAS system 10, i.e., a geometrical pattern A. The optical elements 33 being spherical, they will appear as circles for the tracker device 40. Therefore, the position of the center of the circles can be calculated as the CAS controller 50 may be programmed with the radius or diameter of the optical elements 33.

In the embodiment of FIG. 3B, the geometrical pattern A consists of a triangle defined by the centers of the optical elements 33. In a variant, the triangle is a scalene triangle. Although a triangular geometrical pattern is illustrated, it is contemplated to use other geometrical patterns, such as lines and various polygonal shapes.

The tracker 30 of FIG. 3B may have a calibration file F (FIG. 2). The calibration file F is a digital record of the specific details of the geometrical pattern of the tracker 30 of FIG. 3B, e.g., the length of all three segments and angles between adjacent segments of the triangle A. The calibration file F may be part of a read-only memory of the CAS system 10, or may be retrievable. For this purpose, the tracker device 30 may have an identifier, for the calibration file F specific to the model of the tracker 30 of FIG. 3B to be retrieved. In a variant, the calibration file F may not be modified. The calibration file F may be based on a manufacturer 3D model of the tracker 30.

The calibration file F may include other data, such as a geometrical relation between the tracker 30 and the instrument T that supports it, in instances in which the instruments T come with a dedicated tracker 30. Therefore, an example of geometrical relation that may be part of the calibration file F includes a position of a working tip T1, a position and orientation of an axis, etc, of the instrument T relative to the geometrical patterns A, B, C (if A, B and C are present). As an alternative, the geometrical relation between the instrument T and the tracker 30 may be defined during a calibration step intraoperatively or peri-operatively. The calibration file F may be regarded as a permanent calibration, i.e., one that can be used repeatedly for the tracker 30.

In FIGS. 1 and 2, the tracker device 40 is shown as being embodied by an image capture device, capable of illuminating its environment. In a variant, the tracker device 40 may have two (or more) points of view, such that triangulation can be used to determine the position of the tracker devices 30 in space, i.e., the coordinate system of the CAS system 10. The tracker device 40 may emit light, or use ambient light, to observe the trackers 30 from its points of view, so as to determine a position of the trackers 30 relative to itself. By knowing the geometry of the arrangements of trackers 30, the tracker device 40 can produce navigation data enabling the locating of objects within the coordinate system of the CAS system 10. In an embodiment, the tracker device 40 is of the type known as the Polaris products by Northern Digital Inc. The tracker device 40 may form the complementary part of the CMM function of the CAS system 10, with the trackers 30 on the robot base 20A for example.

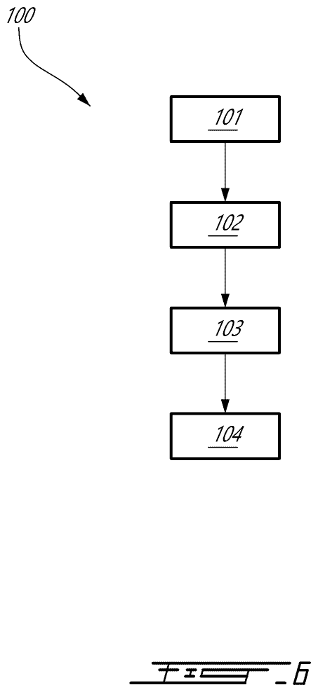

Referring to FIG. 2, the CAS controller 50 is shown in greater detail relative to the other components of the CAS system 10. The CAS controller 50 has a processor unit 51 and a non-transitory computer-readable memory 52 communicatively coupled to the processing unit 51 and configured for executing computer-readable program instructions executable by the processing unit 51 to perform some functions, such as tracking the patient tissue and tools, using the position and orientation data from the robot 20 and the readings from the tracker device 40. Accordingly, as part of the operation of the CAS controller 50, the computer-readable program instructions may include an operating system that may be viewed by a user or operator as a GUI (e.g., with the display of FIG. 5) on one or more of the interfaces of the CAS system 10. It is via this or these interfaces that the user or operator may interface with the CAS system, be guided by a surgical workflow that may include the calibration described herein, obtain navigation data, etc. The CAS controller 50 may also control the movement of the robot arm 20A via the robot controller module 70, of robot arm 20A is present. The CAS system 10 may comprise various types of interfaces I/F, for the information to be provided to the operator. The interfaces I/F may include and/or screens including wireless portable devices (e.g., phones, tablets), audio guidance, LED displays, head-mounted display for virtual reality, augmented reality, mixed reality, among many other possibilities. For example, the interface I/F comprises a graphic-user interface (GUI) operated by the system 10. The CAS controller 50 may also display images captured pre-operatively, or using cameras associated with the procedure (e.g., 3D camera, laparoscopic cameras, tool mounted cameras), for instance to be used in the collaborative/cooperative control mode of the system 10, or for visual supervision by the operator of the system 10, with augmented reality for example. The CAS controller 50 may drive the robot arm 20A, in performing the surgical procedure based on the surgery planning achieved pre-operatively, or in maintaining a given position and orientation to support a tool, if the robot arm 20A is present. The CAS controller 50 may run various modules, in the form of algorithms, code, non-transient executable instructions, etc, in order to operate the CAS system 10 in the manner described herein. The CAS controller 50 may be part of any suitable processor unit, such as a personal computer or computers including laptops and desktops, tablets, server, etc.

The tracking module 60 may be a subpart of the CAS controller 50, or an independent module or system. The tracking module 60 receives the readings from the tracker device 40 and the position and orientation data from the robot 20 (if present). The tracking module 60 may hence determine the relative position of the objects in a referential system. The tracking module 60 may also be provided with models of the objects to be tracked. For example, the tracking module 60 may track bones and tools, and hence may use virtual bone models and tool models, and the tool models may be used in the calibration, as explained below. The bone models may be acquired from pre-operative imaging (e.g., MRI, CT-scans), for example in 3D or in multiple 2D views, including with 2D X-ray to 3D bone model technologies. The virtual bone models may also include some image processing done preoperatively, for example to remove soft tissue or refine the surfaces that will be exposed and tracked. The virtual bone models may be of greater resolution at the parts of the bone that will be tracked during surgery, such as the knee articulation in knee surgery. The bone models may also carry additional orientation data, such as various axes (e.g., longitudinal axis, mechanical axis, etc). The bone models may therefore be patient specific. It is also considered to obtain bone models from a bone model library, with the data obtained from the video images used to match a generated 3D surface of the bone with a bone from the bone atlas. The virtual tool models may be provided by the tool manufacturer, or may also be generated in any appropriate way so as to be a virtual 3D representation of the tool(s). For example, the tool models may be generated intraoperatively using cameras that are part of the CAS system 10 (e.g., tracker device 40).

Additional data may also be available, such as tool orientation (e.g., axis data and geometry). By having access to bone and tool models, the tracking module 60 may obtain additional information, such as the axes related to bones or tools.

Still referring to FIG. 2, the CAS controller 50 may have the robot controller 70 integrated therein, if a robot is present. However, the robot controller 70 may be physically separated from the CAS controller 50, for instance by being integrated into the robot 20 (e.g., in the robot base 20B). The robot controller 70 is tasked with powering and/or controlling the various joints of the robot arm 20A. The robot controller 70 may also optionally calculate robot movements of the robot arm 20A, so as to control movements of the robot arm 20A autonomously in some instances, i.e., without intervention from the CAS controller 50. There may be some force feedback provided by the robot arm 20A to avoid damaging the bones, to avoid impacting other parts of the patient or equipment and/or personnel. The robot controller 70 may perform actions based on a surgery planning. The surgery planning may be a module programmed specifically for any given patient, according to the parameters of surgery desired by an operator such as an engineer and/or surgeon. The parameters may include geometry of selected, planned bone cuts, planned cut depths, sequence or workflow of alterations with a sequence of surgical steps and tools, tools used, etc.

In an embodiment, the tracking module 60 uses a tracker 30 on the bone B or other body portion or OR table to obtain the orientation of the bone B in the coordinate system, and locates the bone B using other methods, such as obtaining the position and orientation of a probing tool such as the registration pointer T of FIG. 3A or FIG. 3B, or by image processing, as different options. Stated differently, the bone B may be fixed on the OR table and the system 10 may rely on trackers 30 fixed to the OR table to optically track the bone B, and point registration steps may be performed to locate the bone B in the referential system of the surgery.

As observed herein, the trackers 30/the tracker device 40 and the tracking from the robot controller 70 may be complementary and/or redundant tracking technologies. The position and orientation of the surgical tool T calculated by the tracking module 60 using optical tracking (i.e., 30 and 40) may be redundant over the tracking data provided by the robot controller 70 and/or the CAS controller 50 and its embedded robot arm sensors 25, referred to as maneuvering data for the robot arm 20A. However, the redundancy may assist in ensuring the accuracy of the tracking of the surgical tool T, and end effector 23, notably when a line of sight is disrupted.

In a variant, the calibrating may include cross-validating the optical tracking data with position data obtained from the robot arm sensors 25. For example, during the calibrating, the instrument T may be supported by the end effector 23 of the robot arm 20A, such that the position and orientation of the instrument T is independently determinable from the robot arm sensors 25 via forward kinematics. The tracking module 60 may compare the position and/or orientation of the tracker 30 as determined by optical tracking with the position and/or orientation of the instrument T as determined from the robot arm sensors 25. A discrepancy between the optically-determined position and the kinematically-determined position may indicate an error in the calibration, which error may be corrected by adjusting values in the calibration file F. This may lead to an update in the calibration file F. In an embodiment, the cross-validation is performed at a plurality of positions and orientations of the instrument T within the workspace of the robot arm 20A, such that calibration errors are characterized across a range of configurations. The cross-validation may be performed as part of the calibrating, or may be performed as a verification step after the calibrating is complete.

The calibration file F may include a geometrical relation (e.g., position and orientation) between the working end of the instrument T and the tracker 30 (whether acquired during calibration tracking or being preprogrammed from tool specifications); specific dimensions of patterns A, B, C (e.g., length of segments and angles between segments), and a geometrical relation between patterns A, B and C, i.e., between the two or more sets 33 of a tracker 30 of FIG. 3A (e.g., set of optical elements 33A, set of optical elements 33B, etc), for instance relative to a reference point on the instrument; specific dimensions of a pattern such as A, B, C for tracker 30 of FIG. 3A, or pattern A for tracker 30 of FIG. 3B, or for an active tracker. The geometrical relation and/or pattern dimensions may be created, adjusted, corrected, based on the difference. In a variant, there is no calibration file F, or the calibration file F is generated through the various steps described below. In another variant, the user may be prompted to recalibrate the instrument T and tracker 30.

In a variant, the calibration file F may be stored on a remote server and retrieved via a network connection. For example, the CAS controller 50 may communicate with a calibration server over a wired or wireless network, including a local area network, a wide area network, or the Internet. Upon identifying the tracker 30 (e.g., by a unique identifier), the CAS controller 50 may transmit a request to a calibration server for the calibration file F associated with the identified tracker 30. The calibration server may maintain a database of calibration files F for a plurality of trackers 30, which database may be updated as calibration files F are generated or modified. When the calibrating results in an updated calibration file F, the CAS controller 50 may transmit the updated calibration file F to the calibration server for storage in the database. In this manner, a calibration performed on a first CAS system 10 may be made available to a second CAS system 10 when the same tracker 30 is subsequently used with the second CAS system 10. The calibration server may be operated by a healthcare institution, a device manufacturer, or a third-party service provider, as examples among others. Access to the calibration files F on the server may be controlled by authentication credentials and/or encryption to maintain data security.

Thus, if the tracker 30 is one that corresponds to the multifaceted tracker of FIG. 3A, the calibration file F may include a geometrical relation between the working end of the instrument T and all three of the geometrical patterns A, B, C (e.g., the triangular patterns of 31A, of 31B and of 31C), but also the geometrical relation between the two or more triangular patterns A, B and C (three shown in FIG. 3A), and the dimensions of the patterns A, B and C. The calibration approach may result in the corrections being made to only one of geometrical relations between the working end of the instrument T and the triangular patterns, or two of the three (if there are three), or all of them. The calibration approach could also be performed to generate a non-existent calibration file F, for one time use, or for subsequent uses.

Thus, the calibrating of the instrument T may optionally include making corrections in the geometrical relation between working end of the instrument T and the tracker 30, and/or between the geometrical patterns A, B and/or C, and/or to the dimensions of one or more of the patterns A, B and/or C, if a calibration file F is available. Once calibrating is achieved, the instrument T may subsequently be used to perform actions on the bone, with the CAS system 10 using the data obtained via the calibration to track the instrument T. For example, if the instrument T is a registration pointer as in FIG. 3A and FIG. 3B, surfacic data may be acquired to generate bone models of the bone.

FIG. 5 shows a graphical user interface (GUI) of the type output by the CAS controller 50 and displayed on an interface I/F. The GUI is one specifically showing an on-screen display to assist in calibration steps to address the issue of variability in the relative position, orientation and/or size of the patterns A, B and C. In a variant, the display of the GUI as in FIG. 5 is one that may assist a user in manipulating the instrument T according to a sequence of movements for the calibration of the tracker 30. The GUI may have a realtime display GUI1 that guides the user in positioning the instrument T with tracker 30 within a preferred calibration volume, illustrated as a circle as one option among others. The calibration volume may be with a preferred distance range of the tracker device 40. As observed from GUI1, the instrument T may be defined by a symbol, or like geometric figure, which must be positioned within the calibration volume. The instrument T must be entirely within the calibration volume during the calibration steps.

The GUI may also include another realtime display GUI2, that may guide the user in performing given movements of the instrument T with tracker 30. The movements may include manipulations according to which the instrument T is held in a given orientation, for the tracker device 40 to see at least two of the patterns of the tracker 30 simultaneously. For example, the GUI2 may guide the user in rotating the tracker 30 relative to its center to reach a suitable orientation, in which patterns A and B, or B and C, or A and C, are concurrently visible. In a variant, the suitable orientation may have all three patterns A, B and C, visible concurrently, if possible and if three patterns A, B and C are present. The GUI2 may provide guidance to ensure that sufficient information is acquired by the tracker device 40 for the calibration file F to be updated or generated. Accordingly, the CAS system 10 may complete the calibration when the geometry of the patterns A, B and C is set and recorded, and/or the geometric relation between the geometrical patterns A, B and C is known and set, as in FIG. 4B, in spite of a multitude of potential relations that may be present as in FIG. 4A.

In a variant, the CAS system 10 may automatically detect a degradation in tracking accuracy and prompt recalibration of the instrument T. For example, the tracking module 60 may monitor one or more tracking quality metrics during the tracking of the instrument T. The tracking quality metrics may include a residual error between detected positions of the optical elements 33 and expected positions based on the calibration file F, a consistency metric indicating variation in detected positions over a time window, a confidence score output by the image processing, or a comparison between optical tracking data and an independent position reference such as the robot arm sensors 25. When a tracking quality metric falls below a predetermined threshold, or when a trend in the tracking quality metric indicates degradation over time, the CAS controller 50 may automatically generate a prompt on the graphical user interface indicating that recalibration is recommended. In an embodiment, the CAS controller 50 may suspend tracking-dependent functions until recalibration is performed. The predetermined threshold may be user-configurable or may be set based on the requirements of the surgical procedure being performed.

In a variant, the existing calibration file F may be retrieved and used, based on the identity of the tracker 30 and/or instrument T. For example, by retrieving the calibration file F, the CAS processor 50 may benefit from preestablished geometrical patterns A, B and/or C to facilitate a recognition of the geometrical patterns A, B and/or C when processing images/video feed from the tracker device 40. If corrections and/or adjustments are then required, the use of the preestablished geometrical patterns A, B and/or C may be used as a baseline for the corrections.

The calibration may further include establishing a geometrical relation between the tracker 30 and a working end of the instrument T, recording the dimensions of the patterns A, B and/or C. In a variant, the geometrical relation between the tracker 30 and a working end of the instrument T is present in the calibration file F and may be used once the dimensions of and the geometrical relation between the patterns A, B and C in the calibration file F is corrected (if corrected). In another variant, a step is performed to locate the working end of the instrument T. Among possibilities, image processing is performed using depth cameras to generate a 3D model of the instrument, which 3D model is tied to the geometrical patterns A, B and C for FIG. 3A, or the geometrical pattern A for FIG. 3B. The guidance of the GUI may be used for the system 10 to obtain images enabling the generating of a 3D model. In another variant, the working end of the instrument T may be positioned in a suitable jig. The jig may be tracked in space via appropriate trackers (e.g., tracker 30), and may have a portion configured to receive the working end of the instrument T in a predetermined manner. In another variant, specific movements are performed for the instrument T, to calculate a position and/or orientation of the working end of the instrument T relative to the tracker 30 on the instrument. For example, if the instrument T is a registration pointer as in FIG. 3A and of FIG. 3B, a tip of the instrument T may be received against a surface, with circular motions performed while the tip of the instrument T serves as pivot for the circular motions. By image processing the video feed from the circulation motions, the CAS controller 50 may determine the position of the tip of the registration pointer, and may locate a central axis of its shaft. In these various scenarios, an existing calibration file F, if available, may be used as baseline information.

Now that the various components of the CAS system 10 have been described, a contemplated procedure performed with the CAS system 10 or with a similar CAS system is set forth, with reference to a flow chart 100 illustrative of a method for calibrating a tracker in computer-assisted surgery is shown in FIG. 6, and is an example of a procedure that may be performed by the CAS controller 50 and/or other parts of the CAS system 10 of the present disclosure. For example, the method 100, may be computer-readable program instructions in the non-transitory computer-readable memory 52 for example, and executable by the processing unit 51 communicatively coupled to the processing unit 51.

According to 101, in an optional step, a calibration file for the instrument with tracker is retrieved, if available. The calibration file may be equivalent to the calibration file F of FIG. 2, as described above.

According to 102, one or more images of an instrument having a tracker thereon is obtained intra-operatively, the tracker having optical elements thereon arranged in a given pattern. The tracker may be the tracker 30 of FIG. 3A, the tracker 30 of FIG. 3B, or any other optically tracked tracker, including an active tracker with LEDs. Intra-operatively may include peri-operatively, i.e., before the patient is operated on. Intra-operatively may include obtaining the one or more images the day of the surgery, in preparation for a surgical procedure. The day of the surgery may include a time period of less than 24 hours before the patient is operated on. One or more images may include a video feed, i.e., a plurality of images obtained over a time period.

The given pattern may be defined by three or more optical elements of the tracker, and the given pattern may be a scalene triangle. The tracker may be a multifaceted tracker such as in FIG. 3A, the multifaceted tracker having two or more sets of three optical elements, each of the two sets forming a geometrical pattern, such as A, B and C, wherein the at least one image includes simultaneously two ore more of the sets of three optical elements, i.e., patterns A and B, B and C, A and C, or A, B and C may be viewed simultaneously in the image. Recording the given pattern may include adjusting values of the calibration file, if a calibration file is present as per 101.

To assist in obtaining the one or more images in 102, a display may be generated and output on a graphical user interface showing a movement required to orient and/or position the instrument with the tracker to obtain said at least one image. Likewise, a display may be generated and output on a graphical user interface showing a position of the instrument with the tracker relative to a calibration volume in which the recording of image and calibrating occurs, to guide a user as to where the tracker should be for recoding the image.

According to 103, the instrument is calibrated by image processing the one or more images of 102, to record the given pattern relative to the instrument. Calibrating the instrument includes image processing the one or more images including the two or more sets of three optical elements to record a geometric relation between the two or more sets of three optical elements, in the case of a multifaceted tracker such as that shown in FIG. 3A. Calibrating the instrument includes image processing the one or more images to record a dimension of at least one of the sets of three optical elements.

Calibrating the instrument according to 103 may also include recording a geometrical relation between the tracker and a working end of the instrument. As a variant, this may include recording the geometrical relation between the tracker and the working end of the instrument, by obtaining a model of the instrument and merging the model with the one or more images. Recording the geometrical relation between the tracker and the working end of the instrument may be performed by obtaining images of the instrument during a given sequence of movement.

In a variant, calibrating the instrument according to 103 may include image processing using a machine learning model. For example, a trained neural network may be used to identify the optical elements 33 in the one or more images, and to determine the positions of the optical elements 33 relative to one another and/or relative to the instrument T. The machine learning model may be trained on a dataset of images of trackers 30 captured at various orientations, distances, and lighting conditions, for example with ground truth labels indicating the positions of the optical elements 33. The machine learning model may be a convolutional neural network, a vision transformer, or other suitable architecture for image analysis. In an embodiment, the machine learning model outputs predicted centroid positions for each detected optical element 33, which predicted positions are then used to calculate the geometric relations between the optical elements 33 and/or between the tracker 30 and the working end of the instrument T. The use of a machine learning model may improve robustness to variations in lighting, partial occlusions, or degraded visibility of the optical elements 33. The machine learning model may be executed by the processing unit 51, or by a dedicated inference accelerator communicatively coupled to the CAS controller 50.

In a variant, the calibrating according to 103 may include cross-validating the optical tracking data with position data obtained from the robot arm sensors 25, if the instrument T is supported by the end effector 23 of the robot arm 20A. Thus, the position and orientation of the instrument T is independently determinable from the robot arm sensors 25 via forward kinematics. The the position and/or orientation of the tracker 30 as determined by optical tracking may be compared with the position and/or orientation of the instrument T as determined from the robot arm sensors 25. A discrepancy between the optically-determined position and the kinematically-determined position may indicate an error in the calibration, which error may be corrected by adjusting values in the calibration file F. In an embodiment, the cross-validation is performed at a plurality of positions and orientations of the instrument T within the workspace of the robot arm 20A, such that calibration errors are characterized across a range of configurations. The cross-validation may be performed as part of the calibrating according to 103, or may be performed as a verification step after the calibrating is complete.

According to 104, the instrument is tracked with the tracker while being used to perform actions on the bone, after the calibrating, by obtaining images of the given pattern of optical elements. 104 may also include continuously tracking the instrument with optical tracking. 104 may also include outputting the tracking data, and this may be in the form of images on an interface, numerical data, etc. The tracking occurs in real-time or quasi real-time, i.e., the tracking values are continuously updated at a frequency that may be faster than a reaction time of a human operator, for example.

Tracking the instrument optically after the calibrating may include tracking the instrument using a first of the at least two sets of optical elements, and switching to tracking the instrument using a second of the at least two sets of optical elements when a line of sight between the first of the at least two sets of optical element and a tracking device is disrupted, when the tracker 30 is a multifaceted tracker as in FIG. 3A.

The calibration sequence and calibrating (e.g., 101 to 104, or 102 to 104) may be repeated at least a second time. The obtaining, calibrating and tracking may be performed intraoperatively. The calibration file, if present, may be updated with the calibrating, for subsequent use.

In a variant, the method 100 may continuously monitor and automatically detect a degradation in tracking accuracy. Once degradation is detected, the method 100 may include prompting recalibration of the instrument T, for example by performing 103 another time. For example, the method 100 may include monitoring one or more tracking quality metrics during the tracking of the instrument T according to 104, hence the monitoring and detection of degradation may be part of 104. The tracking quality metrics may include a residual error between detected positions of the optical elements 33 and expected positions based on the calibration file F, a consistency metric indicating variation in detected positions over a time window, a confidence score output by the image processing, or a comparison between optical tracking data and an independent position reference such as the robot arm sensors 25. When a tracking quality metric falls below a predetermined threshold, or when a trend in the tracking quality metric indicates degradation over time, the method 100 may automatically generate a prompt on the graphical user interface indicating that recalibration is recommended. In an embodiment, the method 100 may include suspending tracking-dependent functions until recalibration is performed. The predetermined threshold may be user-configurable or may be set based on the requirements of the surgical procedure being performed.

The system 10 may thus be generally described as being for tracking an instrument in a robotized computer-assisted surgery, and may include: a processing unit; and a non-transitory computer-readable memory communicatively coupled to the processing unit and comprising computer-readable program instructions executable by the processing unit for:

### EXAMPLE

The following example can stand on its own, or can be combined in different permutations, combinations, with one or more of other examples.

Example 1: A system for tracking an instrument in computer-assisted surgery, comprising: a processing unit; and a non-transitory computer-readable memory communicatively coupled to the processing unit and comprising computer-readable program instructions executable by the processing unit for: obtaining at least one image of an instrument, the instrument having a multifaceted tracker thereon, the multifaceted tracker having at least two sets of three optical elements, each of the two sets forming a geometrical pattern, wherein the at least one image includes at least two of the at least two sets of three optical elements; calibrating the instrument by image processing the at least one image including the at least two of the at least two sets of three optical elements to record a geometric relation between the at least two sets of three optical elements; and tracking the instrument optically after the calibrating by obtaining images of a single one of the at least two sets of three optical elements.

Example 2: a method for tracking an instrument in computer-assisted surgery, comprising: obtaining intraoperatively at least one image of an instrument having a tracker thereon, the tracker having optical elements thereon arranged in a given pattern; calibrating the instrument by image processing the at least one image to record the given pattern relative to the instrument; and tracking the instrument with the tracker optically after the calibrating, by obtaining images of the given pattern of optical elements.

In Example 3, the subject matter of Example 2 may include the tracker being a multifaceted tracker, the multifaceted tracker having at least two sets of three optical elements, each of the two sets forming a geometrical pattern, wherein the at least one image includes at least two of the at least two sets of three optical elements, and wherein calibrating the instrument includes image processing the at least one image including the at least two of the at least two sets of three optical elements to record a geometric relation between the at least two sets of three optical elements.

In Example 4, the subject matter of Example 3 may include calibrating the instrument by including image processing the at least one image including the at least one of the at least two sets of three optical elements to record a dimension of at least one of the sets of three optical elements.

In Example 5, the subject matter of Example 4 may include that the multifaceted tracker has three sets of three optical elements, and wherein obtaining at least one image of an instrument includes obtaining an image including the three sets of three optical elements.

In Example 6, the subject matter of Example 4 may include that the multifaceted tracker has three sets of three optical elements, and wherein obtaining at least one image of an instrument includes obtaining at least a first image including a first and a second of the sets of three optical elements; obtaining at least a second image including a second and a third of the sets of three optical elements; and obtaining at least a third image including a first and a third of the sets of three optical elements.

In Example 7, the subject matter of Example 2 may include calibrating the instrument by further including recording a geometrical relation between the tracker and a working end of the instrument.

In Example 8, the subject matter of Example 7 may include recording the geometrical relation between the tracker and the working end of the instrument by performing by obtaining a model of the instrument and merging the model with the at least one image.

In Example 9, the subject matter of Example 7 may include recording the geometrical relation between the tracker and the working end of the instrument by obtaining images of the instrument during a given sequence of movement.

In Example 10, the subject matter of Example 2 may include a calibration file retrieved for the instrument, and wherein recording the given pattern includes adjusting values of the calibration file.

In Example 11, the subject matter of Example 2 may include a display outputting on a graphical user interface showing a movement required to orient and/or position the instrument with the tracker to obtain said at least one image.

In Example 12, the subject matter of Example 2 may include a display outputting on a graphical user interface showing a position of the instrument with the tracker relative to a calibration volume in which the calibrating occurs.

In Example 13, the subject matter of Example 2 may include that the tracker is a multifaceted tracker, the multifaceted tracker having at least two sets of three optical elements, each of the two sets forming a geometrical pattern, wherein the at least one image includes at least two of the at least two sets of three optical elements, and wherein tracking the instrument optically after the calibrating includes tracking the instrument using a first of the at least two sets of optical elements, and switching to tracking the instrument using a second of the at least two sets of optical elements when a line of sight between the first of the at least two sets of optical element and a tracking device is disrupted.

In Example 14, the subject matter of Example 2 may include obtaining at least one image by obtaining a video feed.

In Example 15, the subject matter of Example 2 may include the obtaining, calibrating and tracking performed intraoperatively.

In Example 16, the subject matter of Example 2 may include a degradation in tracking accuracy that may be automatically detected, prompting a recalibration.

1. A system for tracking an instrument in computer-assisted surgery, comprising:
a processing unit; and
a non-transitory computer-readable memory communicatively coupled to the processing unit and comprising computer-readable program instructions executable by the processing unit for:
obtaining intraoperatively at least one image of an instrument having a tracker thereon, the tracker having optical elements thereon arranged in a given pattern;
calibrating the instrument by image processing the at least one image to record the given pattern relative to the instrument; and
tracking the instrument with the tracker optically after the calibrating, by obtaining images of the given pattern of optical elements.

2. The system according to claim 1, wherein the tracker has three optical elements thereon, and the given pattern is a scalene triangle.

3. The system according to claim 1, wherein the tracker is a multifaceted tracker, the multifaceted tracker having at least two sets of three optical elements, each of the two sets forming a geometrical pattern, wherein the at least one image includes at least two of the at least two sets of three optical elements.

4. The system according to claim 3, wherein calibrating the instrument includes image processing the at least one image including the at least two of the at least two sets of three optical elements to record a geometric relation between the at least two sets of three optical elements.

5. The system according to claim 3, wherein calibrating the instrument includes image processing the at least one image including the at least one of the at least two sets of three optical elements to record a dimension of at least one of the sets of three optical elements.

6. The system according to claim 3, wherein the multifaceted tracker has three sets of three optical elements.

7. The system according to claim 6, wherein obtaining at least one image of an instrument includes obtaining an image including the three sets of three optical elements.

8. The system according to claim 6, wherein obtaining at least one image of an instrument includes obtaining at least a first image including a first and a second of the sets of three optical elements; obtaining at least a second image including a second and a third of the sets of three optical elements; and obtaining at least a third image including a first and a third of the sets of three optical elements.

9. The system according to claim 1, wherein calibrating the instrument further includes recording a geometrical relation between the tracker and a working end of the instrument.

10. The system according to claim 9, wherein recording the geometrical relation between the tracker and the working end of the instrument is performed by obtaining a model of the instrument and merging the model with the at least one image.

11. The system according to claim 9, wherein recording the geometrical relation between the tracker and the working end of the instrument is performed by obtaining images of the instrument during a given sequence of movement.

12. The system according to claim 1, including retrieving a calibration file for the instrument, and wherein recording the given pattern includes adjusting values of the calibration file.

13. The system according to claim 1, further including outputting a display on a graphical user interface showing a movement required to orient and/or position the instrument with the tracker to obtain said at least one image.

14. The system according to claim 1, further including outputting a display on a graphical user interface showing a position of the instrument with the tracker relative to a calibration volume in which the calibrating occurs.

15. The system according to claim 2, wherein tracking the instrument optically after the calibrating includes tracking the instrument using a first of the at least two sets of optical elements, and switching to tracking the instrument using a second of the at least two sets of optical elements when a line of sight between the first of the at least two sets of optical element and a tracking device is disrupted.

16. The system according to claim 1, wherein obtaining at least one image includes obtaining a video feed.

17. The system according to claim 1, wherein the optical elements are retroreflective members, and wherein the three optical elements of a first of the sets are in a first orientation, the three optical elements of a second of the sets are in a second orientation.

18. The system according to claim 1, wherein the obtaining, calibrating and tracking are performed intraoperatively.

19. The system according to claim 1, further including automatically detecting a degradation in tracking accuracy, and prompting a recalibration.

20. A method for tracking an instrument in computer-assisted surgery, comprising:
obtaining intraoperatively at least one image of an instrument having a tracker thereon, the tracker having optical elements thereon arranged in a given pattern;
calibrating the instrument by image processing the at least one image to record the given pattern relative to the instrument; and
tracking the instrument with the tracker optically after the calibrating, by obtaining images of the given pattern of optical elements.
